SEQUENCE LISTING (57 sequences)

SEQ ID NO: 1 - DNA, 23 bp, Artificial Sequence
  Synthesized
cgccaatggtgttaacacatagg

SEQ ID NO: 2 - DNA, 110 bp, Artificial Sequence
  Synthesized
gacgtatggtgttgggtaaatccgggaggacatttgcatgtgaagccgccaatggtgttaacacgtaggaactggcagttgtgttgcttggttgcacactcatcaagatc

SEQ ID NO: 3 - DNA, 23 bp, Artificial Sequence
  Synthesized
ccgccaatggtgttaacacgtag

SEQ ID NO: 4 - DNA, 687 bp, Artificial Sequence
  Synthesized
atgagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctacagcccatgcggcgaatgtagtagggccatcactgaattcctgtcaaggtatccccacgtcactctgtttatttacatcgcaaggctgtaccaccacgctgaccccgagaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaa

SEQ ID NO: 5 - DNA, 687 bp, Artificial Sequence
  Synthesized
atgagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctatagcccatgcggcgaatgtagtagggccatcactgaattcctgtcaaggtatccccacgtcactctgtttatttacatcgcaaggctgtaccaccacgctgacccccgcaatcgacaaggcctggaagatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaa

SEQ ID NO: 6 - DNA, 687 bp, Artificial Sequence
  Synthesized
atgagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctacagcccatgcggcgaatgtagtagggccatcactgaattcctgtcaaggtatccccacgtcactctgtttatttacatcgcaaggctgtaccaccacgctgaccccgagaatcgacaaggcctggaggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaa

SEQ ID NO: 7 - DNA, 8532 bp, Artificial Sequence
  Synthesized
atatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatgagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctacagcccatgcggcgaatgtagtagggccatcactgaattcctgtcaaggtatccccacgtcactctgtttatttacatcgcaaggctgtaccaccacgctgaccccgagaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaaagcggcagcgagactcccgggacctcagagtccgccacacccgaaagtgataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgactctggtggttctactaatctgtcagatattattgaaaaggagaccggtaagcaactggttatccaggaatccatcctcatgctcccagaggaggtggaagaagtcattgggaacaagccggaaagcgatatactcgtgcacaccgcctacgacgagagcaccgacgagaatgtcatgcttctgactagcgacgcccctgaatacaagccttgggctctggtcatacaggatagcaacggtgagaacaagattaagatgctctctggtggttctcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatc

SEQ ID NO: 8 - DNA, 8532 bp, Artificial Sequence
  Synthesized
atatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatgagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctatagcccatgcggcgaatgtagtagggccatcactgaattcctgtcaaggtatccccacgtcactctgtttatttacatcgcaaggctgtaccaccacgctgacccccgcaatcgacaaggcctggaagatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaaagcggcagcgagactcccgggacctcagagtccgccacacccgaaagtgataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgactctggtggttctactaatctgtcagatattattgaaaaggagaccggtaagcaactggttatccaggaatccatcctcatgctcccagaggaggtggaagaagtcattgggaacaagccggaaagcgatatactcgtgcacaccgcctacgacgagagcaccgacgagaatgtcatgcttctgactagcgacgcccctgaatacaagccttgggctctggtcatacaggatagcaacggtgagaacaagattaagatgctctctggtggttctcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatc

SEQ ID NO: 9 - DNA, 8604 bp, Artificial Sequence
  Synthesized
atatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagacccaagctggctagcaccatgggacctaagaaaaagaggaaggtgtctagagactacaaggatgacgacgataaaggatccatgagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctacagcccatgcggcgaatgtagtagggccatcactgaattcctgtcaaggtatccccacgtcactctgtttatttacatcgcaaggctgtaccaccacgctgaccccgagaatcgacaaggcctggaggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaaagcggcagcgagactcccgggacctcagagtccgccacacccgaaagtgataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgactctggtggttctactaatctgtcagatattattgaaaaggagaccggtaagcaactggttatccaggaatccatcctcatgctcccagaggaggtggaagaagtcattgggaacaagccggaaagcgatatactcgtgcacaccgcctacgacgagagcaccgacgagaatgtcatgcttctgactagcgacgcccctgaatacaagccttgggctctggtcatacaggatagcaacggtgagaacaagattaagatgctctctggtggttctcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatc

SEQ ID NO: 10 - DNA, 8532 bp, Artificial Sequence; identical to SEQ ID NO: 7
  Synthesized

SEQ ID NO: 11 - DNA, 2791 bp, Artificial Sequence
  Synthesized
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatatcggatccctaatacgactcactataggcgccaatggtgttaacacatgttttagagctagaaatagcaagttaaaataaggctagtccgttatcaacttgaaaaagtggcaccgagtcggtgctttttttaaagggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcggccgccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 12 - DNA, 4950 bp, Artificial Sequence
  Synthesized
ggtaccgattagtgaacggatctcgacggtatcgatcacgagactagcctcgagcggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctacgtgttaacaccattgggttttagagctagaaatagcaagttaaaataaggctagtccgttatcaacttgaaaaagtggcaccgagtcggtgctttttttaaagaattctcgacctcgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggccgcggctcgagggggttggggttgcgccttttccaaggcagccctgggtttgcgcagggacgcggctgctctgggcgtggttccgggaaacgcagcggcgccgaccctgggactcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggaaacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcggtacctttaagaccaatgacttacaaggcagctgtagatcttagccactttctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 13 - DNA, 2791 bp, Artificial Sequence
  Synthesized
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatatcggatccctaatacgactcactataggctacgtgttaacaccattgggttttagagctagaaatagcaagttaaaataaggctagtccgttatcaacttgaaaaagtggcaccgagtcggtgctttttttaaagggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcggccgccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 14 - DNA, 24 bp, Artificial Sequence
  Synthesized
taggcgccaatggtgttaacacat

SEQ ID NO: 15 - DNA, 24 bp, Artificial Sequence
  Synthesized
aaacatgtgttaacaccattggcg

SEQ ID NO: 16 - DNA, 24 bp, Artificial Sequence
  Synthesized
accgctacgtgttaacaccattgg

SEQ ID NO: 17 - DNA, 24 bp, Artificial Sequence
  Synthesized
aaacccaatggtgttaacacgtag

SEQ ID NO: 18 - DNA, 24 bp, Artificial Sequence
  Synthesized
taggctacgtgttaacaccattgg

SEQ ID NO: 19 - DNA, 24 bp, Artificial Sequence
  Synthesized
aaacccaatggtgttaacacgtag

SEQ ID NO: 20 - DNA, 20 bp, Artificial Sequence
  Synthesized
tttcccatgattccttcata

SEQ ID NO: 21 - DNA, 24 bp, Artificial Sequence
  Synthesized
cgccagggttttcccagtcacgac

SEQ ID NO: 22 - DNA, 24 bp, Artificial Sequence
  Synthesized
tctcgcgcgtttcggtgatgacgg

SEQ ID NO: 23 - DNA, 31 bp, Artificial Sequence
  Synthesized
aaaaaaagcaccgactcggtgccactttttc

SEQ ID NO: 24 - DNA, 20 bp, Artificial Sequence
  Synthesized
actcaccaatgcaggacgta

SEQ ID NO: 25 - DNA, 20 bp, Artificial Sequence
  Synthesized
agctgcttcatagggtcagc

SEQ ID NO: 26 - DNA, 19 bp, Artificial Sequence
  Synthesized
gctgaagtctccacccacc

SEQ ID NO: 27 - DNA, 19 bp, Artificial Sequence
  Synthesized
tgtctctccttgccttttg

SEQ ID NO: 28 - DNA, 20 bp, Artificial Sequence
  Synthesized
tcaagggacaggagtaggca

SEQ ID NO: 29 - DNA, 20 bp, Artificial Sequence
  Synthesized
ttggggcaggaggttttgtt

SEQ ID NO: 30 - DNA, 19 bp, Artificial Sequence
  Synthesized
atcttaatcagggccttga

SEQ ID NO: 31 - DNA, 19 bp, Artificial Sequence
  Synthesized
gccttcattccatcaactg

SEQ ID NO: 32 - DNA, 20 bp, Artificial Sequence
  Synthesized
caggttcgtgtcgcagtagc

SEQ ID NO: 33 - DNA, 19 bp, Artificial Sequence
  Synthesized
ctgtgttgccagcacgaaa

SEQ ID NO: 34 - DNA, 20 bp, Artificial Sequence
  Synthesized
tggtagtggttggtgacact

SEQ ID NO: 35 - DNA, 20 bp, Artificial Sequence
  Synthesized
cgttacattgggaagcggaa

SEQ ID NO: 36 - DNA, 20 bp, Artificial Sequence
  Synthesized
ggattcaacatagattggaa

SEQ ID NO: 37 - DNA, 18 bp, Artificial Sequence
  Synthesized
cccgtttacacattgcta

SEQ ID NO: 38 - DNA, 20 bp, Artificial Sequence
  Synthesized
ttctagtaggtgaaaaaggg

SEQ ID NO: 39 - DNA, 19 bp, Artificial Sequence
  Synthesized
ttggacaccacatagacag

SEQ ID NO: 40 - DNA, 22 bp, Artificial Sequence
  Synthesized
tattattgctaaaccgaaacca

SEQ ID NO: 41 - DNA, 18 bp, Artificial Sequence
  Synthesized
agcccctcacccactcat

SEQ ID NO: 42 - DNA, 20 bp, Artificial Sequence
  Synthesized
agaggcttgcgaaggacatc

SEQ ID NO: 43 - DNA, 20 bp, Artificial Sequence
  Synthesized
atttggtctagggcagaggc

SEQ ID NO: 44 - DNA, 20 bp, Artificial Sequence
  Synthesized
attattcacaagttatggta

SEQ ID NO: 45 - DNA, 18 bp, Artificial Sequence
  Synthesized
taaccctcttctttgtaa

SEQ ID NO: 46 - DNA, 23 bp, Artificial Sequence
  Synthesized
aagggactgtttttgtcctgtca

SEQ ID NO: 47 - DNA, 23 bp, Artificial Sequence
  Synthesized
gtgaaaccaccatgacatgaagt

SEQ ID NO: 48 - DNA, 20 bp, Artificial Sequence
  Synthesized
gtcatacttggccagggtcc

SEQ ID NO: 49 - DNA, 20 bp, Artificial Sequence
  Synthesized
cccacgtgagctggctaaaa

SEQ ID NO: 50 - DNA, 20 bp, Artificial Sequence
  Synthesized
tgatcagcatgtggagcctg

SEQ ID NO: 51 - DNA, 20 bp, Artificial Sequence
  Synthesized
gaagtcagccaggagccatt

SEQ ID NO: 52 - DNA, 18 bp, Artificial Sequence
  Synthesized
gagttaggagtgggaagg

SEQ ID NO: 53 - DNA, 21 bp, Artificial Sequence
  Synthesized
acaaaggacagtaatgaagag

SEQ ID NO: 54 - DNA, 20 bp, Artificial Sequence
  Synthesized
tttgcctccttgattccccc

SEQ ID NO: 55 - DNA, 20 bp, Artificial Sequence
  Synthesized
gtggatggtgtggaggtgag

SEQ ID NO: 56 - DNA, 22 bp, Artificial Sequence
  Synthesized
cgataaagggatcagtcactaa

SEQ ID NO: 57 - DNA, 19 bp, Artificial Sequence
  Synthesized
gctccaggtccacaaacac

(19) United States
(12) Patent Application Publication
(10) Pub. No.: US 2021/0198699 A1
(43) Pub. Date: Jul. 1, 2021

(54) KIT FOR REPARING FBN1T7498C MUTATION, COMBINATION FOR MAKING AND REPAIRING MUTATION, AND METHOD OF REPAIRING THEREOF

(71) Applicant: SHANGHAITECH UNIVERSITY, Shanghai (CN)

(72) Inventors: Xingxu HUANG, Shanghai (CN); Guanglei LI, Shanghai (CN); Jianan LI, Shanghai (CN)

(73) Assignee: SHANGHAITECH UNIVERSITY, Shanghai (CN)

(21) Appl. No.: 16/470,247

(22) PCT Filed: Jul. 25, 2018
(86) PCT No.: PCT/CN2018/096968

§ 371 Date: Jun. 17, 2019

(30) Foreign Application Priority Data
Jun. 1, 2018 (CN) 201810560722.0

(51) Int. Cl. C12N 15/90; C07K 14/47; C12N 15/113; C12N 9/22

(52) U.S. Cl. CPC C12N 15/907; C12N 9/22; C12N 15/113; C07K 14/47

(57) ABSTRACT

A reagent and a method for repairing an FBN1^{T7498C} mutation by using base editing. A kit for efficiently repairing the FBN1^{T7498C} mutation is characterized by comprising a base editor and a repair re-sgRNA directed to the FBN1^{T7498C} site. A base editing technology is used to repair the mutation of FBN1^{T7498C} through a precise CT single base mutation, thereby providing a method for treating a Marfan syndrome caused by such mutation.

### BACKGROUND

### Technical Field

The present invention relates to the field of gene repair, and more particularly to a method for repairing a Marfan syndrome-associated FBN1^{T7498C} mutation by using base editing.

### Related Art

To date, nearly 10,000 genetic diseases have been identified in medicine, placing a huge burden on families and society, yet only about 6% of genetic diseases are currently treatable (Austin and Dawkins, 2017). Diagnosing and treating genetic diseases has become an important research topic in medicine. The development of high-throughput sequencing technology has made the diagnosis of genetic diseases easier. Although pre-implantation genetic diagnosis (PGD) can block the occurrence of some genetic diseases, it is necessary to develop more effective genetic treatment means for some genetic diseases such as homozygous mutations (Dunbar et al., 2018).

The gene editing technology, especially CRISPR/Cas9, has been widely applied to gene manipulation and can be applied to accurately repair disease-causing mutations (Komor et al., 2017). At the same time, the technology has also shown great advantages in genetic editing of human embryos, suggesting its clinical value in the treatment of human genetic diseases (Kang et al., 2016). However, due to the limitation of the ethics and efficiency of treatment and the existence of off-target, there is still much room for improvement in the application of gene editing in human embryos (Ruzo and Brivanlou, 2017). CRISPR/Cas9-mediated gene editing is to allow an sgRNA (single guided RNA) to guide the Cas9 protein through target sequence complementation to position and cleave a double-stranded DNA, resulting in double-strand DNA breaks (DSB); in the absence of a template, non-homologous end joining (NHEJ) repair occurs to cause a frameshift mutation, resulting in gene knockout; in the presence of the template, homology-directed repair (HDR) is used to achieve gene knockin; the efficiency of HDR is low (integration rarely occurs), and a non-homologous end joining mechanism is easy to generate random insertions and deletions (indel), so that new bases may be introduced randomly near a breakpoint, thereby causing inaccurate gene editing (Hsu et al., 2014). In addition, CRISPR/Cas9-mediated gene editing always has some off-target effects [Gorski et al., 2017].

A recently developed base editor based on a nicking enzyme nCas9 is added with rat cytosine deaminase APOBEC1. By means of the base editor, C on a target site can be converted to uracil without cleaving a DNA double strand (Komor et al., 2016). Thereafter, by DNA replication or repair, uracil is converted to thymine (T), thereby achieving a conversion from C to T, and similarly, a single base G can also be converted to A therethrough. Since DSB is caused without cleaving DNA, the formed indel is less than 1%, and the gene editing achieved is more accurate. The base editor has been successfully applied to in vivo base editing to achieve CT mutations in mice. We also use BE3 to achieve efficient editing of target sites in discarded human embryos.

The Marfan syndrome is an autosomal dominant genetic disease, accounting for 0.2% of the world's population. It mainly causes abnormal development of human connective tissues. At present, the sudden death of many well-known athletes is related to the Marfan syndrome (Arbustini et al., 2005). Although some patients can be treated surgically, there is still a risk of disease in their offspring, and the genetic treatment of mutations causing the disease will be a fundamental method. The mutation of the FBN1 gene has been previously shown to be the main cause of the Marfan syndrome. Therefore, whether an effective and safe treatment method can be found is the direction we pursue. The present invention aims to find a method for more efficiently and safely repairing Marfan syndrome-associated mutations and repairing the mutations at the human embryonic stage, thereby reducing the incidence and the huge social burden of the disease.

### REFERENCES

Arbustini, E., Grasso, M., Ansaldi, S., Malattia, C., Pilotto, A., Porcu, E., Disabella, E., Marziliano, N., Pisani, A., Lanzarini, L., et al. (2005). Identification of sixty-two novel and twelve known FBN1 mutations in eighty-one unrelated probands with Marfan syndrome and other fibrillinopathies. Human mutation 26, 494.
Austin, C. P., and Dawkins, H. J. S. (2017). Medical research: Next decade's goals for rare diseases. Nature 548, 158.
Dunbar, C. E., High, K. A., Joung, J. K., Kohn, D. B., Ozawa, K., and Sadelain, M. (2018). Gene therapy comes of age. Science 359.
Hsu, P. D., Lander, E. S., and Zhang, F. (2014). Development and applications of CRISPR-Cas9 for genome engineering. Cell 157, 1262-1278.
Kang, X., He, W., Huang, Y., Yu, Q., Chen, Y., Gao, X., Sun, X., and Fan, Y. (2016). Introducing precise genetic modifications into human 3PN embryos by CRISPR/Cas-mediated genome editing. Journal of assisted reproduction and genetics 33, 581-588.
Komor, A. C., Badran, A. H., and Liu, D. R. (2017). CRISPR-Based Technologies for the Manipulation of Eukaryotic Genomes. Cell 168, 20-36.
Komor, A. C., Kim, Y. B., Packer, M. S., Zuris, J. A., and Liu, D. R. (2016). proceduremable editing of a target base in genomic DNA without double-stranded DNA cleavage. Nature 533, 420-424.
Ruzo, A., and Brivanlou, A. H. (2017). At Last: Gene Editing in Human Embryos to Understand Human Development. Cell Stem Cell 21, 564-565.

### SUMMARY

The object of the present invention is to provide a kit and a method for efficiently repairing an FBN1^{T7498C} mutation.

To achieve the above object, the present invention provides a kit for efficiently repairing an FBN1^{T7498C} mutation, which is characterized by comprising a base editor and a repair re-sgRNA directed to an FBN1^{T7498C} site.

Preferably, the base editor is one of BE3, YE1-BE3, YE2-BE3 or YEE-BE3.

Preferably, a sequence of the repair re-sgRNA directed to the FBN1^{T7498C} site is SEQ ID NO. 3.

The present invention also provides a combination for making a mutation and repairing the mutation, which is characterized by comprising at least one of a mutated mt-sgRNA designed according to the FBN1^{T7498C} site and a correspondingly mutated ssODN, a repair re-sgRNA directed to the FBN1^{T7498C} site, and a base editor.

The present invention also provides a method for repairing a mutation by using base editing, which is characterized by comprising: in an FBN1T⁷⁴⁹⁸C-containing mutated cell, using a repair re-sgRNA directed to the FBN1^{T7498C} site to guide a base editor to a mutation site to perform base editing repair and collect transfected cells.

Preferably, the FBN1T⁷⁴⁹⁸C-containing mutated cell is an HEK293T cell or an embryonic cell.

Preferably, a method for constructing the FBN1^{T7498C}-containing mutated cell comprises: designing a mutated mt-sgRNA and a correspondingly mutated ssODN according to the FBN1^{T7498C} site; constructing an expression vector of mt-sgRNA, and allowing Cas9 protein and transcribed mt-sgRNA in vitro to form an RNP complex to be electrotransfected into HEK293T cells with ssODN, and performing flow sorting on single cells and identifying an FBN1^{T7498C}-containing mutated cell line.

Preferably, the repair re-sgRNA directed to the FBN1^{T7498C} site is obtained by designing the FBN1^{T7498C} site and constructing a U6 activated and/or T7 activated expression vector.

Preferably, the method for repairing the Marfan disease mutation by base editing also comprises:

detecting the repair efficiency by Sanger sequencing; injecting mRNA and re-sgRNA of the base editor into the human embryonic cell containing the FBN1^{T7498C} mutation, detecting the repair efficiency in the embryos, and detecting the on-target and off-target efficiency by high-throughput sequencing.

Preferably, a sequence of the mt-sgRNA is SEQ ID NO. 1, a sequence of ssODN is SEQ ID NO. 2, and a sequence of re-sgRNA is SEQ ID NO. 3.

Preferably, a method for obtaining the embryos is to inject a sperm containing the FBN1^{T7498C} site mutation into a normal oocyte by ICSI, or to inject a normal sperm into an egg containing the FBN1^{T7498C} mutation site by ICSI, or to inject the sperm containing the FBN1^{T7498C} site mutation into an egg containing the FBN1^{T7498C} mutation site to obtain a heterozygous or homozygous mutated embryo containing this site.

The present invention also provides a method for repairing a Marfan disease mutation by base editing, comprising: designing a mutated mt-sgRNA according to the FBN1^{T7498C} site and a corresponding mutated ssODN; constructing a T7 activated expression vector of mt-sgRNA, allowing a Cas9 protein and the transcribed mt-sgRNA in vitro to form an RNP to be bound to ssODN and electrotransfect 293T cells, and performing flow sorting on single cells and identifying an FBN1^{T7498C}-containing homozygous mutated cell line; designing a repair re-sgRNA according to the FBN1^{T7498C} site, and respectively constructing U6 activated and T7 activated expression vectors; in the made homozygous mutated cell line, using U6 activated re-sgRNA to guide the base editor to the mutation site, collecting the transfected cells after 3 days, and detecting the repair efficiency by Sanger sequencing; injecting mRNA and re-sgRNA of BE3 into human embryonic cells containing the site mutation, detecting the repair efficiency in the embryos, and detecting the on-target and off-target efficiency by high-throughput sequencing.

The present invention proves the high efficiency and safety of the method at two different levels of cells and embryos by using the base editing technology.

The mutation of FBN1 is the main cause of the Marfan syndrome. Its incidence rate is about 0.2%, and the genetically complete repair of this mutation will be the most effective measure to treat the disease. The base editor provides a method for precisely changing DNA, namely, a method for converting C to T.

In the present invention, the mutated human embryos are repaired by using the base editor as a novel base editing tool. The safety and efficacy of the method will first be verified at two levels of cells and embryos. In terms of cells, the inventors will use a homologous recombination method based on CRISPR/Cas9 and ssODN to make a cell line containing the FBN1^{T7498C} mutation, and then use the base editor to be bound to a suitable sgRNA to repair the mutation at this site. After verifying the safety and efficacy of the system, the transcribed mRNA and sgRNA are injected into the human embryos containing the FBN1^{T7498C} mutation, the embryos are collected after three days, and the repair efficiency and the off-target situation are detected by a deep sequencing method.

In the present invention, the base editing technology is used to repair the FBN1^{T7498C} mutation through the precise CT single base mutation, thereby providing an efficient and safe method for treating the Marfan syndrome caused by such mutation.

### BRIEF DESCRIPTION OF THE DRAWINGS

FIG. 1 shows the confirmation of mutation sites in samples from patients with a Marfan syndrome. (A) is sampled from blood, (B) is sampled from semen.

FIG. 2 shows the making of mutated cell lines in 293T cells by means of Cas9/sgRNA bound to ssODN. (A) is a mode diagram of making a mutation and repairing a mutation in cells. (B) shows a design of mutation type sgRNA and ssODN for the FBN1 gene to make corresponding mutations. (C) is T7EN1 enzyme digestion identification on transfection efficiency after transfecting cells. (D) is flow sorting of twenty-two single cell clones, and sanger sequencing confirmation of mutation types of cells. (E) is a sanger sequencing peak map of homozygous mutations.

FIG. 3 shows off-target detection sorting of the mutated sgRNA. (A) is the analysis of potential off-target sites of the mutated sgRNA by software, and detection of corresponding off-target sites and detection of off-target by T7EN1for the homozygous mutated cell lines. (B) is sanger sequencing detection on mutations.

FIG. 4 shows the repair of mutated cell lines by using base editing. (A) is design of a corresponding repair sgRNA at the mutation site and repair by using base editing. (B) is sequencing detection on the mutation situation after cell transfection. (C) is TA clone identification of repair type of Figure B. (D) is a sanger sequencing peak map after complete repair.

FIG. 5 shows repair of mutated cell lines by using YE1-BE3, YEE-BE3 and BE3.

FIG. 6 shows off-target detection of repair sgRNA. (A) is enzyme digestion detection of off-target by using T7EN1. (B) is confirmation of off-target situation by using sanger sequencing.

FIG. 7 shows repair of mutated human embryos. (A) is a schematic diagram of human embryo repair by using base editing. (B) shows an embryonic state after the embryos containing the mutations are injected with the relevant RNA. (C) is the genotype of representative embryos injected with the repair sgRNA. (D) is the genotype of representative embryos injected with a random sgRNA. (E) is genotypic analysis of repaired embryos and control embryos by high-throughput sequencing.

FIG. 8 shows genotypes detected of the repaired embryos and control embryos used. (A) is the genotype of repaired embryos. (B) is the genotype of control embryos.

FIG. 9 shows detection of off-target of the repair sgRNA by using high-throughput sequencing in the repaired embryos.

### DETAILED DESCRIPTION

The examples of the present invention will be clearly and completely described with reference to embodiments. It is apparent that the described embodiments are only intended to illustrate a part of the embodiments of the present invention and are not to be construed as limiting the scope of the present invention. If no specific conditions are specified in the embodiments, the embodiments are carried out according to conventional conditions or the conditions recommended by manufacturers. If reagents or instruments used are not labeled with the specific manufacturers, they are considered to be conventional products that can be purchased commercially.

The above is only the preferred embodiments of the present invention, and is not intended to limit the present invention. Any modifications, equivalent substitutions, improvements, etc. within the spirit and scope of the present invention should be included in the scope of protection of the present invention.

First, different versions of BE3, namely YE1-BE3, YE2-BE3, YEE-BE3, and BE3, were constructed. The original version BE3 was purchased from Addgene (73021).

1. Synthesis of Different Types of APOBEC1

The above four kinds of BE3 differ only in the coding frame of rAPOBEC1, and sequences of YE1-rAPOBEC1 (SEQ ID NO. 4), YE2-rAPOBEC1 (SEQ ID NO. 5), and YEE-rAPOBEC1 (SEQ ID NO. 6) were synthesized by Sangon Biotech (http://www.sangon.com/). The two ends of the synthesized fragments were respectively ligated with enzyme digestion sites of NotI and SmaI. The synthesized fragments were cloned onto a normally used pmd19t vector (TAKARA: 6013).

2. Enzyme Digestion and Purification of Vectors

BE3 and the above three synthesized vectors were enzyme-digested with NotI (NEB: R0189L) and SmaI (NEB: R0141L). The system was as follows: 6 uL of Buffer (NEB: R0189L), 2 ug of plasmid, 1 μL of NotI, 1 μL of SmaI and ddH₂O complemented to 60 μL. After samples were mixed, the enzyme digestion was carried out overnight at a temperature of 37° C.

The enzyme-digested product was recovered by 1% of agarose gel (Axygen: AP-GX-250G). BE3 recovered the backbone vector of large fragments, and the synthesized vector recovered small fragment vector of APOBEC1. The recovery was carried out according to the instructions for use of a kit (Axygen: AP-PCR-250G). The concentrations of the recovered fragments were detected by Nanodrop 2000.

3. Ligation, Transformation and Plasmid Extraction of Vectors

The recovered backbone vector and the APOBEC1 fragment were ligated by the following system: 1 μL of T4 ligation buffer (NEB: M0202L), 20 ng of backbone vector, 50 ng of APOBEC1 fragment, 0.5 μL of T4 ligase (NEB: M0202L), and ddH₂O complemented to 10 μL. Ligation was carried out overnight at a temperature of 16° C. The transformation step comprises: taking 20 μL of competent cells (TransGen: CD201) and thawing on ice, mixing 2 μL of a ligation product with the competent cells, placing the mixture on ice for 20 minutes, performing heat-shocking at a temperature of 42° C. for 60 seconds, placing the mixture on ice for 2 minutes, adding 400 μL of a resuscitation LB medium (MDBio: L001-1 kg) and shaking on a shaker for 30 minutes, taking 70 μL of an ampicillin-coated plate (50 μg/ml), and culturing in a 37° C. incubator for 14 hours.

Monoclonal bacteria were selected and cultured in 4 ml of a liquid LB medium on a large scale, and the plasmids (Axygene: AP-MN-P-250G) were extracted after 14 hours. The step comprises: centrifuging a bacterial solution at a speed of 4,000 rpm for 10 minutes, and pouring a supernatant medium out; adding 350 μL of a buffer S1, blowing off the thalli, and transferring to a 2 ml centrifuge tube; adding 250 μL of a buffer S2 and putting upside down for 8 times; adding 250 μL of a buffer S3, putting upside down and mixing evenly for 6 times to produce a precipitate; centrifuging at a speed of 12,000 rpm for 10 minutes, taking a supernatant and performing column chromatography; centrifuging for 1 minute, pouring a waste solution out, adding 500 μL of W1, centrifuging for one minute, and pouring a waste solution out; adding 750 μL of W2, centrifuging, and pouring a supernatant out; adding 500 μL of W2, centrifuging, and pouring a supernatant out; idling for 1 minute; adding 50 μL of an eluate, keeping stand for 2 minutes, and centrifuging. The obtained plasmids were subjected to concentration detection, 10 μL of plasmids were taken for sequencing, and positive plasmids were stored at a temperature of −20° C.

Finally, four types of BE3 were constructed as follows:

(1) YE1-BE3, SEQ ID NO. 7;

(2) YE2-BE3, SEQ ID NO. 8;

(3) YEE-BE3, SEQ ID NO. 9;

(4) BE3, SEQ ID NO. 10.

In the case of performing the mutation site repair below, any one of the above BE3 may be used, preferably (1) or (4).

### Embodiment 1

In this embodiment, a mutation FBN1^{T7498C} mutated cell line was made by using Cas9/sgRNA bound to ssODN on a cell line, and the mutated cell line was repaired by using a base editor (FIG. 2).

1.1 Plasmid Construction

Near a mutation site, a mutated mt-sgRNA (SEQ ID NO. 1) was designed and oligos were synthesized. The upstream sequence was 5′-taggCGCCAATGGTGTTAACACAT-3′ (SEQ ID NO. (14)), the downstream sequence was 5′-aaacATGTGTTAACACCATTGGCG-3′ (SEQ ID NO. (15)), and the upstream and downstream sequences were annealed by a procedure (95° C., 5 min; 95° C. to 85° C. at−2° C./s; 85° C. to 25° C. at−0.1° C./s; hold at 4° C.) and ligated to a PUC57-T7sgRNA vector (addgene: 51132) linearized with BsaI (NEB: R0539L). The linearization system was as follows: 2 μg of PUC57-T7sgRNA, 6 μL of buffer (NEB: R0539L), 2 μL of BsaI, and ddH₂O complemented to 60 μL. The enzyme digestion was carried out overnight at a temperature of 37° C. A homologous template ssODN (SEQ ID NO. 2) used was synthesized by means of PAGE purification by Sangon Biotech Company (http://www.sangon.com/). At the same time, near the mutation site, according to the characteristics of base editing, a repair re-sgRNA (SEQ ID NO. 3) was designed, and oligos were synthesized, the upstream sequence was 5′-accgCTACGTGTTAACACCATTGG-3′ (SEQ ID NO. 16), and the downstream sequence was 5′-aaacCCAATGGTGTTAACACGTAG-3′ (SEQ ID NO. 17). The upstream and downstream sequences were annealed by a procedure (95° C., 5 min; 95° C.−85° C. at−2° C./s; 85° C.−25° C. at−0.1° C./s; hold at 4° C.), and ligated to a PGL3-U6sgRNA vector linearized with BsaI (NEB: R0539L). At the same time, an upstream primer: 5′-taggCTACGTGTTAACACCATTGG-3″ (SEQ ID NO. 18) and a downstream primer: 5′-aaacCCAATGGTGTTAACACGTAG-3′ (SEQ ID NO. 19) were synthesized and ligated to a linearized PUC57-T7sgRNA vector by annealing. The annealing procedure, the linearization system and procedure were as above. The ligation system was as follows: 1 μL of T4 ligation buffer (NEB: M0202L), 20 ng of linearized vector, 5 μL of annealed oligo fragment (10 μM), 0.5 μL of T4 ligase (NEB: M0202L), and ddH₂O complemented to 10 μL. Ligation was carried out overnight at a temperature of 16° C. The ligated vector was subjected to transformation, bacterium selection and identification, the U6 vector as the identification primer was the upstream sequence: 5′-TTTCCCATGATTCCTTCATA-3′ (SEQ ID NO. 20), and the downstream sequence was the downstream sequence of the corresponding oligo. The T7 vector was the upstream sequence: 5′-CGCCAGGGTTTTCCCAGTCACGAC-3′ (SEQ ID NO. 21), and the downstream sequence was the downstream sequence of the corresponding oligo. For the positive clone, the bacteria were shaken, the plasmids were extracted (Axygene: AP-MN-P-250G), and the concentration was measured to be ready for use. The obtained mutated plasmids were named as mt-T7-sgRNA (SEQ ID NO. 11), re-U6-sgRNA (SEQ ID NO. 12) and re-T7-sgRNA (SEQ ID NO. 13).

1.2 In Vitro Transcription of sgRNA

A fragment containing sgRNA was amplified by using the constructed PUC57-T7sgRNA as a template, and the primers used were F: 5′-TCTCGCGCGTTTCGGTGATGACGG-3′ (SEQ ID NO. 22), R: 5′-AAAAAAAGCACCGACTCGGTGCCACTTTTTC-3′ (SEQ ID NO. 23). The amplification system was as follows: 25 μL of 2×buffer (Vazyme: P505), 1 μL of dNTP, 2 μL of F (10 pmol/μL), 2 μL of R (10 pmol/μL), 1 ng of template, 0.5 μL of DNA polymerase (Vazyme: P505), and ddH₂O complemented to 50 μL. The amplified PCR product was purified by the following steps of adding 4 μL of RNAsecure (Life: AM7005) per 100 μL volume; the treatment was performed at 60° C. for 15 minutes; adding three times volume of PCR-A (Axygen: AP-PCR-250G), performing column chromatography, centrifuging, centrifuging at a speed of 12,000 rpm for 1 minute, adding 500 μL of W2, and centrifuging for 1 minute; idling for 1 minute, and adding 20 μL of RNA-free water to perform elution.

The steps of transcribing by using an in vitro transcription kit (Ambion, Life Technologies, AM1354) were as follows. The reaction system was as follows: 1 μL of reaction buffer, 1 μL of enzyme mix, 1 μL of A, 1 μL of T, 1 μL of G, 1 μL of C, 800 ng of template, and H₂O complemented to 10 μL. The above system was mixed evenly and reacted at a temperature of 37° C. for 5 hours. 1 μL of DNase was added and reacted at a temperature of 37° C. for 15 minutes. The transcribed sgRNA was recovered by a recovery kit (Ambion, Life Technologies, AM1908) by the following steps: adding 90 μL of an Elution solution to the reaction volume of a product in the above step and transplanting the mixture to a 1.5 ml EP tube; adding 350 μL of a Binding solution to mix evenly; adding 250 μL of absolute ethanol to mix evenly; performing column chromatography; centrifuging at a speed of 10,000 rpm for 30 seconds, and pouring a waste solution out; adding 500 μL of a Washing solution, centrifuging at a speed of 10,000 rpm for 30 seconds, and pouring a waste solution out; idling for 1 minute; replacing a collection tube, and adding 100 μL of an Elution solution to perform elution; adding 10 μL of ammonium acetate (Ambion, Life Technologies, AM1908) to mix evenly; adding 275 μL of absolute ethanol to mix evenly; keeping stand at a temperature of −20° C. for 30 minutes while preparing 70% ethanol and keeping stand at a temperature of −20° C.; centrifuging at a speed of 13,000 rpm for 15 minutes at a temperature of 4° C.; discarding a supernatant, and adding 500 μL of 70% ethanol; centrifuging for 5 minutes, drawing away a waste solution, and air-drying for 5 minutes; adding 20 μL of water to dissolve; and taking 1 μL and measuring the concentration.

1.3 Cell Culture and Electrotransfection

(1) Taking HEK293T cells (purchased from ATCC) as an example, the culture and transfection of eukaryotic cells were carried out in the present invention as follows: the HEK293T cells were inoculated and cultured in a DMEM high glucose culture solution (HyClone, SH30022.01B) added with 10% FBS, wherein penicillin (100 U/ml) and streptomycin (100 μg/ml) were contained.

(2) The medium was replaced with an antibiotic-free medium two hours before the transfection, and the transfection was carried out by using an LONZA transfection reagent (SF KIT) according to the instructions, and the cells were counted to be 1×10⁶. Cas9 (Sigma: ESPCAS9PRO-50UG), sgRNA and ssODN were mixed according to a mass of 3 μg, 1.5 μg and 3 μg. The electrotransfection procedure was performed by using DS150, and the electrotransfected cells were cultured in a 6 cm dish for two days.

(3) Single cells were sorted out from the cells by a flow sorter to be cultured. After two weeks, the genotype was identified by splitting decomposition. A lysate comprises the following components: 50 mM KCl, 1.5 mM MgCl₂, 10 mM Tris pH 8.0, 0.5% Nonidet P-40, 0.5% Tween 20, and 100 μg/ml protease K. Cell lines that were homozygously mutated were selected and cultured on a large scale.

(4) For the mutated cell lines, 7 related off-target sites were identified by http://crispr.mit.edu/, https://crispr.cos.uni-heidelberg.de/, as shown in Table 1.

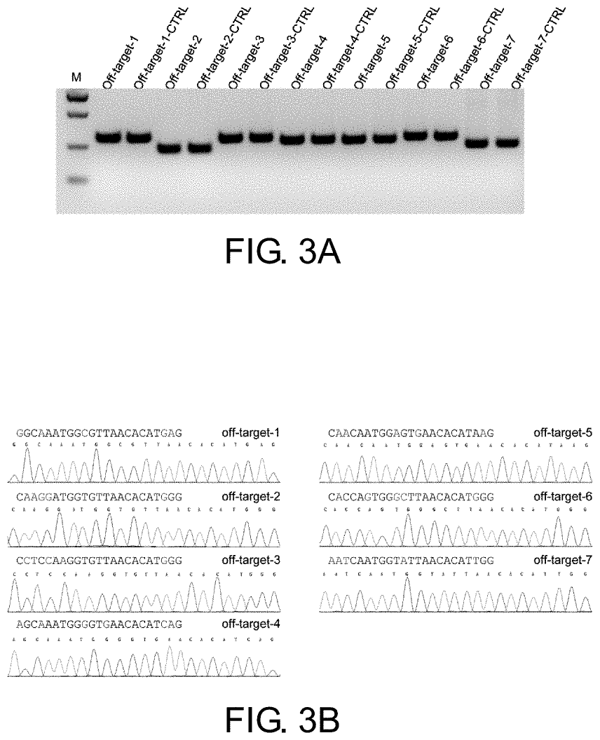

(5) The off-target sites were identified by designing corresponding primers, and the primer sequences were as shown in Table 3. The amplified PCR product was identified by T7EN1 and sequencing, and no off-target was found (FIG. 3).

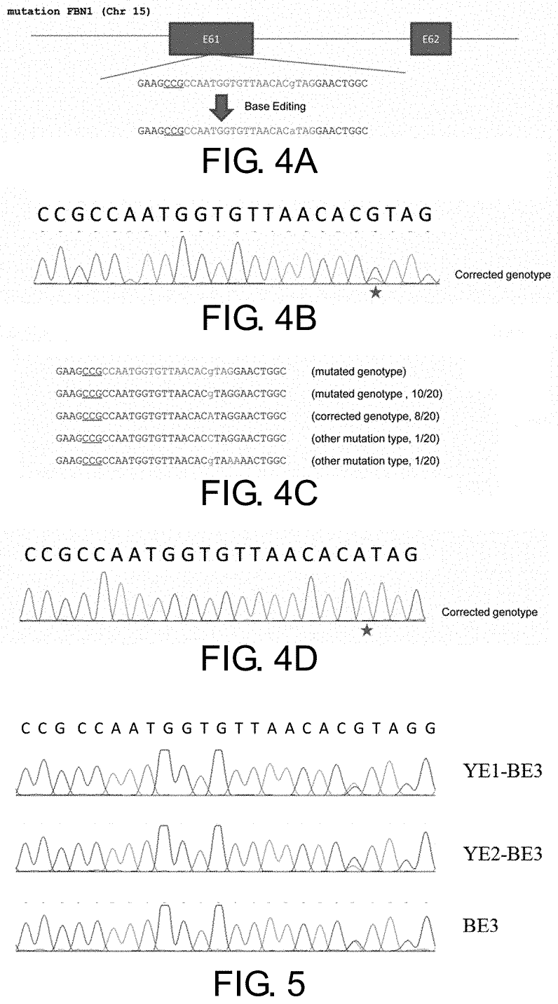

1.4 Repair of Mutated Cell Lines (FIG. 4, FIG. 5)

(1) The mutated cell lines were cultured and transfected in the present invention as follow: HEK293T cells were inoculated and cultured in a DMEM high glucose culture solution (HyClone, SH30022.01B) added with 10% FBS, which contains penicillin (100 U/ml) and streptomycin (100 μg/ml).

(2) The cells were distributed in a 6-pore plate before transfection, and transfected when the density reached 70%-80%.

(3) Transfection was exemplified by lipofection. 2 μg of BE3 or YE1-BE3 or YEE-BE3 plasmid was evenly mixed with 1 μg of re-sgRNApGL3-U6-sgRNA plasmid according to the operation manual of Lipofectamine™ 2000 Transfection Reagent (Invitrogen, 11668-019), and co-transfected into the cells per pore, the solution was replaced after 6-8 hours, and the cells were collected after 72 hours.

(4) The collected cells were firstly subjected to PCR product sequencing, and the repaired peak values were found (FIG. 3). Further, the PCR product was subjected to TA cloning, and in the selected clones, the cloning efficiency of repair reached 50%.

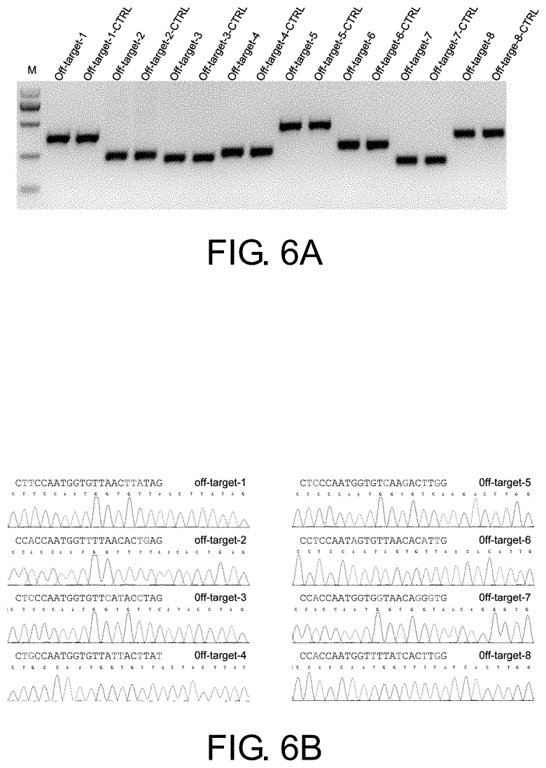

(5) The off-target analysis of the repaired re-sgRNA was carried out. Similarly, 8 potential off-target sites were found, as shown in Table 2 and FIG. 3. Similarly, the potential off-target sites were analyzed and detected (Table 3, FIG. 6), and no off-target phenomenon was found.

TABLE 1
Potential off-target sites of mt-sgRNA

| | | | | | | | | | | | | | | | | | | | | | | | | Location | | | |
|---|---|---|---|---|---|---|---|---|---|---|---|---|---|---|---|---|---|---|---|---|---|---|---|---|---|---|---|
| | | | | | | | | | | | | | | | | | | | | | | | | | | | Positive |
| | | | | | | | | | | | | | | | | | | | | | | | | Chro- | | | and |
| mt- | 20 | 19 | 18 | 17 | 16 | 15 | 14 | 13 | 12 | 11 | 10 | 9 | 8 | 7 | 6 | 5 | 4 | 3 | 2 | 1 | N | G | G | mo- | | | negative |
| sgRNA | C | G | C | C | A | A | T | G | G | T | G | T | T | A | A | C | A | C | g | T | A | G | G | some | Start | End | chains |
| off- | [symbol] | G | C | [symbol] | A | A | T | G | G | [symbol] | G | T | T | A | A | C | A | C | A | T | [symbol] | [symbol] | G | 2 | 200716608 | 200716630 | − |
| target-1 | | | | | | | | | | | | | | | | | | | | | | | | | | | |
| off- | C | [symbol] | [symbol] | [symbol] | [symbol] | A | T | G | G | T | G | T | T | A | A | C | A | C | A | T | [symbol] | G | G | 12 | 45042464 | 45042486 | + |
| target-2 | | | | | | | | | | | | | | | | | | | | | | | | | | | |
| off- | C | [symbol] | [symbol] | C | [symbol] | A | [symbol] | G | G | T | G | T | T | A | A | C | A | C | A | T | [symbol] | G | G | 8 | 71760055 | 71760077 | − |
| target-3 | | | | | | | | | | | | | | | | | | | | | | | | | | | |
| off- | [symbol] | G | C | [symbol] | A | A | T | G | G | [symbol] | G | T | [symbol] | A | A | C | A | C | A | T | [symbol] | [symbol] | G | 18 | 38365508 | 38365530 | − |
| target-4 | | | | | | | | | | | | | | | | | | | | | | | | | | | |
| off- | C | [symbol] | [symbol] | C | A | A | T | G | G | [symbol] | G | T | [symbol] | A | A | C | A | C | A | T | A | [symbol] | G | 4 | 171343505 | 171343527 | + |
| target-5 | | | | | | | | | | | | | | | | | | | | | | | | | | | |
| off- | C | [symbol] | C | C | A | [symbol] | T | G | G | [symbol] | [symbol] | T | T | A | A | C | A | C | A | T | [symbol] | G | G | X | 7778820 | 7778842 | + |
| target-6 | | | | | | | | | | | | | | | | | | | | | | | | | | | |
| off- | [symbol] | [symbol] | [symbol] | C | A | A | T | G | G | T | [symbol] | T | T | A | A | C | A | C | A | T | [symbol] | G | G | 2 | 223099020 | 223099042 | + |
| target-7 | | | | | | | | | | | | | | | | | | | | | | | | | | | |

TABLE 2
Potential off-target sites of re-sgRNA

| | | | | | | | | | | | | | | | | | | | | | | | | Location | | | |
|---|---|---|---|---|---|---|---|---|---|---|---|---|---|---|---|---|---|---|---|---|---|---|---|---|---|---|---|
| | | | | | | | | | | | | | | | | | | | | | | | | | | | Positive |
| | | | | | | | | | | | | | | | | | | | | | | | | Chro | | | and |
| re- | C | C | N | 1 | 2 | 3 | 4 | 5 | 6 | 7 | 8 | 9 | 10 | 11 | 12 | 13 | 14 | 15 | 16 | 17 | 18 | 19 | 20 | mo- | | | negative |
| sgRNA | C | C | G | C | C | A | A | T | G | G | T | G | T | T | A | A | C | A | C | g | T | A | G | some | Start | End | chains |
| off- | C | [symbol] | [symbol] | C | C | A | A | T | G | G | T | G | T | T | A | A | C | [symbol] | [symbol] | [symbol] | T | A | G | 2 | 151200690 | 151200712 | + |
| target-1 | | | | | | | | | | | | | | | | | | | | | | | | | | | |
| off- | C | C | [symbol] | C | C | A | A | T | G | G | T | [symbol] | T | T | A | A | C | A | C | [symbol] | [symbol] | A | G | 4 | 100842267 | 100842289 | + |
| target-2 | | | | | | | | | | | | | | | | | | | | | | | | | | | |
| off- | C | [symbol] | [symbol] | C | C | A | A | T | G | G | T | G | T | T | [symbol] | A | [symbol] | A | C | [symbol] | T | A | G | 7 | 115952661 | 115952683 | + |
| target-3 | | | | | | | | | | | | | | | | | | | | | | | | | | | |
| off- | C | [symbol] | [symbol] | C | C | A | A | T | G | G | T | G | T | T | A | T | T | A | C | T | T | A | [symbol] | 11 | 76525281 | 76525303 | − |
| target-4 | | | | | | | | | | | | | | | | | | | | | | | | | | | |
| off- | C | [symbol] | [symbol] | C | C | A | A | T | G | G | T | G | T | [symbol] | A | A | [symbol] | A | C | [symbol] | T | [symbol] | G | 5 | 76052091 | 76052113 | − |
| target-5 | | | | | | | | | | | | | | | | | | | | | | | | | | | |
| off- | C | C | [symbol] | C | C | A | A | T | [symbol] | G | T | G | T | T | A | A | C | A | C | [symbol] | T | [symbol] | G | 5 | 116017402 | 116017424 | − |
| target-6 | | | | | | | | | | | | | | | | | | | | | | | | | | | |
| off- | C | C | [symbol] | C | C | A | A | T | G | G | T | G | [symbol] | T | A | A | C | A | [symbol] | G | [symbol] | [symbol] | G | 5 | 172090193 | 172090215 | − |
| target-7 | | | | | | | | | | | | | | | | | | | | | | | | | | | |
| off- | C | C | [symbol] | C | C | A | A | T | G | G | T | [symbol] | T | T | A | [symbol] | C | A | C | [symbol] | T | [symbol] | G | 2 | 131478269 | 131478291 | − |
| target-8 | | | | | | | | | | | | | | | | | | | | | | | | | | | |

TABLE 3
Primer sequences used in this project

| Primer names | Sequences (5′-3′) | | Primer uses |
|---|---|---|---|
| FBN1-ON-200-F | ACTCACCAATGCAGGACGTA | SEQ ID | Amplification |
| | | NO. 24 | of target sites |
| FBN1-ON-200-R | AGCTGCTTCATAGGGTCAGC | SEQ ID | |
| | | NO. 25 | |
| FBN1-ON-676-F | GCTGAAGTCTCCACCCACC | SEQ ID | |
| | | NO. 26 | |
| FBN1-ON-676-R | TGTCTCTCCTTGCCTTTTG | SEQ ID | |
| | | NO. 27 | |
| FBN1-mutation-off1-301-F | TCAAGGGACAGGAGTAGGCA | SEQ ID | Amplification |
| | | NO. 28 | of potential |
| FBN1-mutation-off1-301-R | TTGGGGCAGGAGGTTTTGTT | SEQ ID | off-target sites |
| | | NO. 29 | |
| FBN1-mutation-off2-223-F | ATCTTAATCAGGGCCTTGA | SEQ ID | |
| | | NO. 30 | |
| FBN1-mutation-off2-223-R | GCCTTCATTCCATCAACTG | SEQ ID | |
| | | NO. 31 | |
| FBN1-mutation-off3-296-F | CAGGTTCGTGTCGCAGTAGC | SEQ ID | |
| | | NO. 32 | |
| FBN1-mutation-off3-296-R | CTGTGTTGCCAGCACGAAA | SEQ ID | |
| | | NO. 33 | |
| FBN1-mutation-off4-282-F | TGGTAGTGGTTGGTGACACT | SEQ ID | |
| | | NO. 34 | |
| FBN1-mutation-off4-282-R | CGTTACATTGGGAAGCGGAA | SEQ ID | |
| | | NO. 35 | |
| FBN1-mutation-off5-272-F | GGATTCAACATAGATTGGAA | SEQ ID | |
| | | NO. 36 | |
| FBN1-mutation-off5-272-R | CCCGTTTACACATTGCTA | SEQ ID | |
| | | NO. 37 | |
| FBN1-mutation-off6-302-F | TTCTAGTAGGTGAAAAAGGG | SEQ ID | |
| | | NO. 38 | |
| FBN1-mutation-off6-302-R | TTGGACACCACATAGACAG | SEQ ID | |
| | | NO. 39 | |
| FBN1-mutation-off7-252-F | TATTATTGCTAAACCGAAACCA | SEQ ID | |
| | | NO. 40 | |
| FBN1-mutation-off7-252-R | AGCCCCTCACCCACTCAT | SEQ ID | |
| | | NO. 41 | |
| FBN1-BE-OFF1-370-F | AGAGGCTTGCGAAGGACATC | SEQ ID | Amplification |
| | | NO. 42 | of potential |
| FBN1-BE-OFF1-370-R | ATTTGGTCTAGGGCAGAGGC | SEQ ID | off-target sites |
| | | NO. 43 | |
| FBN1-BE-OFF2-247-F | ATTATTCACAAGTTATGGTA | SEQ ID | |
| | | NO. 44 | |
| FBN1-BE-OFF2-247-R | TAACCCTCTTCTTTGTAA | SEQ ID | |
| | | NO. 45 | |
| FBN1-BE-OFF3-235-F | AAGGGACTGTTTTTGTCCTGTCA | SEQ ID | |
| | | NO. 46 | |
| FBN1-BE-OFF3-235-R | GTGAAACCACCATGACATGAAGT | SEQ ID | |
| | | NO. 47 | |
| FBN1-BE-OFF4-262-F | GTCATACTTGGCCAGGGTCC | SEQ ID | |
| | | NO. 48 | |
| FBN1-BE-OFF4-262-R | CCCACGTGAGCTGGCTAAAA | SEQ ID | |
| | | NO. 49 | |
| FBN1-BE-OFF5-448-F | TGATCAGCATGTGGAGCCTG | SEQ ID | |
| | | NO. 50 | |
| FBN1-BE-OFFS-448-R | GAAGTCAGCCAGGAGCCATT | SEQ ID | |
| | | NO. 51 | |
| FBN1-BE-OFF6-296-F | GAGTTAGGAGTGGGAAGG | SEQ ID | |
| | | NO. 52 | |
| FBN1-BE-OFF6-296-R | ACAAAGGACAGTAATGAAGAG | SEQ ID | |
| | | NO. 53 | |
| FBN1-BE-OFF7-208-F | TTTGCCTCCTTGATTCCCCC | SEQ ID | |
| | | NO. 54 | |
| FBN1-BE-OFF7-208-R | GTGGATGGTGTGGAGGTGAG | SEQ ID | |
| | | NO. 55 | |
| FBN1-BE-OFF8-370-F | CGATAAAGGGATCAGTCACTAA | SEQ ID | |
| | | NO. 56 | |
| FBN1-BE-OFF8-370-R | GCTCCAGGTCCACAAACAC | SEQ ID | |
| | | NO. 57 | |

### Embodiment 2

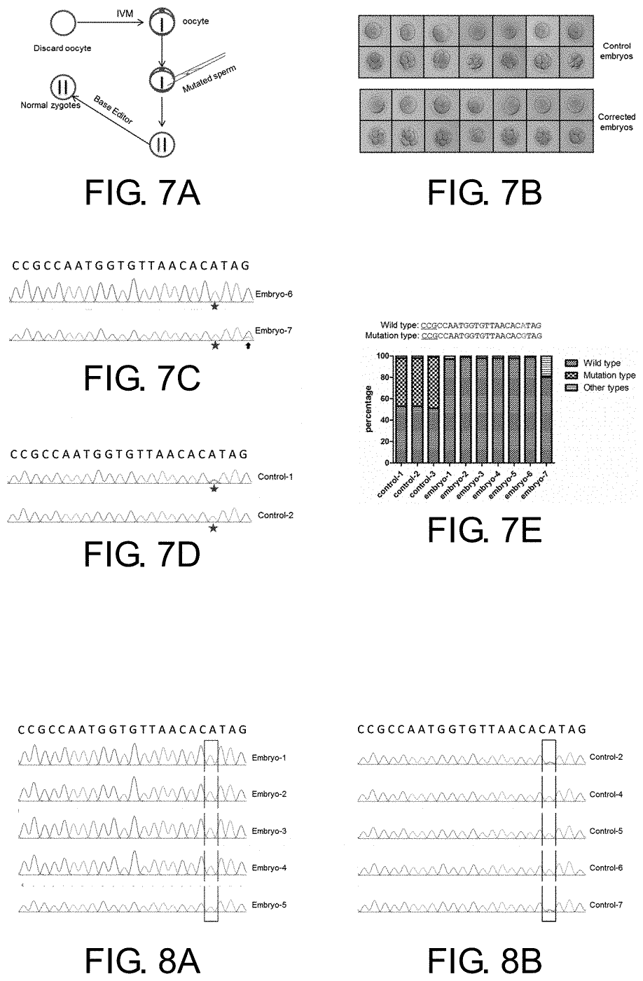

In this embodiment, the mutation was repaired by using a base editor in human embryos (FIG. 7).

2.1 Plasmid Construction

Near the mutation site, according to the characteristics of base editing, a repair re-sgRNA (SEQ ID NO. 3) was designed and oligos were synthesized, the upstream sequence was 5′-taggCTACGTGTTAACACCATTGG-3′ (SEQ ID NO. 18), and the downstream sequence was 5′-aaacCCAATGGTGTTAACACGTAG-3′ (SEQ ID NO. 19). The upstream and downstream sequences were annealed by a procedure (95° C., 5 min; 95° C.−85° C. at−2° C./s; 85° C.−25° C. at−0.1° C./s; hold at 4° C.), and ligated to a PUC57-T7sgRNA vector linearized with BsaI (NEB: R0539L). The linearization system and procedure were as above. The ligation system was as follows: 1 μL of T4 ligation buffer (NEB: M0202L), 20 ng of linearized vector, 5 μL of annealed oligo fragment (10 μM), 0.5 μL of T4 ligase (NEB: M0202L), and ddH₂O complemented to 10 μL. Ligation was carried out overnight at a temperature of 16° C. The ligated vector was subjected to transformation, bacterium selection and identification, the identification primer was the upstream sequence: 5′-CGCCAGGGTTTTCCCAGTCACGAC-3′ (SEQ ID NO. 21), and the downstream sequence was the downstream sequence of the corresponding oligo. For the positive clone, the bacteria were shaken, the plasmids were extracted (Axygene: AP-MN-P-250G), and the concentration was measured to be ready for use.

2.2 In Vitro Transcription of sgRNA

A fragment containing sgRNA was amplified by using the constructed PUC57-T7sgRNA as a template, and the primers used were F: 5′-TCTCGCGCGTTTCGGTGATGACGG-3′ (SEQ ID NO. 22), R: 5′-AAAAAAAGCACCGACTCGGTGCCACTTTTTC-3′ (SEQ ID NO. 23). The amplification system was as follows: 25 μL of 2×buffer (Vazyme: P505), 1 μL of dNTP, 2 μL of F (10 pmol/μL), 2 μL of R (10 pmol/μL), 1 μg of template, 0.5 μL of DNA polymerase (Vazyme: P505), and ddH₂O complemented to 50μL. The amplified PCR product was purified by the following steps of adding 4 μL of RNAsecure (Life: AM7005) per 100 μL volume; the treatment was performed at 60° C. for 15 minutes; adding three times volume of PCR-A (Axygen: AP-PCR-250G) to perform column chromatography, centrifuging, centrifuging at a speed of 12,000 rpm for 1 minute, adding 500 μL of W2, and centrifuging for 1 minute; idling for 1 minute, and adding 20 μL of RNAase-free water to perform elution.

The steps of transcribing by using an in vitro transcription kit (Ambion, Life Technologies, AM1354) were as follows. The reaction system was as follows: 1 μL of reaction buffer, 1 μL of enzyme mix, 1 μL of A, 1 μL of T, 1 μL of G, 1 μL of C, 800 ng of template, and H₂O complemented to 10 μL. The above system was mixed evenly and reacted at a temperature of 37° C. for 5 hours. 1 μL of DNase was added and reacted at a temperature of 37° C. for 15 minutes. The transcribed sgRNA was recovered by a recovery kit (Ambion, Life Technologies, AM1908) by the following steps: adding 90 μL of an Elution solution to the reaction volume of a product in the above step and transplanting to a 1.5 ml EP tube; adding 350 μL of a Binding solution to mix evenly; adding 250 μL of absolute ethanol to mix evenly; performing column chromatography; centrifuging at a speed of 10,000 rpm for 30 seconds, and pouring a waste solution out; adding 500 μL of a Washing solution, centrifuging at a speed of 10,000 rpm for 30 seconds, and pouring a waste solution out; idling for 1 minute; replacing a collection tube, and adding 100 μL of an Elution solution; adding 10 μL of ammonium acetate (Ambion, Life Technologies, AM1908) to mix evenly; adding 275 μL of absolute ethanol to mix evenly; keeping stand at a temperature of −20° C. for 30 minutes while preparing 70% ethanol and keeping stand at a temperature of −20° C.; centrifuging at a speed of 13,000 rpm for 15 minutes at a temperature of 4° C.; discarding a supernatant, and adding 500 μL of 70% ethanol; centrifuging for 5 minutes, drawing away a waste solution, and air-drying for 5 minutes; adding 20 μL of water to dissolve; and taking 1 μL and measuring the concentration.

2.3 In Vitro Transcription of BE3

BE3 enzyme digestion and recovery. This step was to linearize the plasmid BE3. The system was as follows: 10 μg of BE3/YE1-BE3/YE2-BE3/YEE-BE3, 10 μL of buffer I (NEB: R0539L), 4 μL of BbsI (NEB: R0539L), and H₂O complemented to 100 μL. After mixing evenly, the enzyme digestion was carried out overnight at a temperature of 37° C.

Recovery of linearized plasmid. 4 μL of RNAsecure (Life: AM7005) was added to an enzyme-digested product, and reacted at a temperature of 60° C. for 10 minutes; the remaining steps were carried out by using a recovery kit (QIAGEN: 28004), 5 times volume of buffer PB was added and column chromatography was performed; 750 μL of buffer PE was added for centrifugation; the idling was carried out for 1 minute; 10 μL of water was used for elution and the concentration was measured.

In vitro transcription. According to the requirements of a kit (Invitrogen: AM1345), relevant reagents were sequentially added to the system: 1 μg of linearized vector, 10 μL of 2×NTP/ARCA, water complemented to 20 μL, 2 μL of T7 enzyme mix, and 2 μL of 10× reaction buffer. After mixing, the reaction was carried out at a temperature of 37° C. for 2 hours. 1 μL of DNase was added to react for 15 minutes.

Tailing. The transcription product was subjected to a tailing treatment to ensure the stability of the transcribed mRNA. The specific system was as follows: 20 μL of reaction product, 36 μL of H₂O, 20 μL of 5×E-PAP buffer, 10 μL of 25 mM MnCl₂, 10 μL of ATP solution, and 4 μL of PEP. The reaction system was mixed evenly and then reacted at a temperature of 37° C. for 30 minutes.

Recovery. The recovery was carried out by using a recovery kit (QIAGEN: 74104). The steps were as follows: adding 350 μL of buffer RLT to the reaction product in the above step; adding 250 μL of absolute ethanol, performing column chromatography and centrifuging; adding 500 μL of RPE, centrifuging, adding 500 μL of RPE, and centrifuging; idling, and adding 30 μL of water to elute. After the concentration was measured, it was stored at a temperature −80° C.

2.4 Obtaining of Mutated Embryos

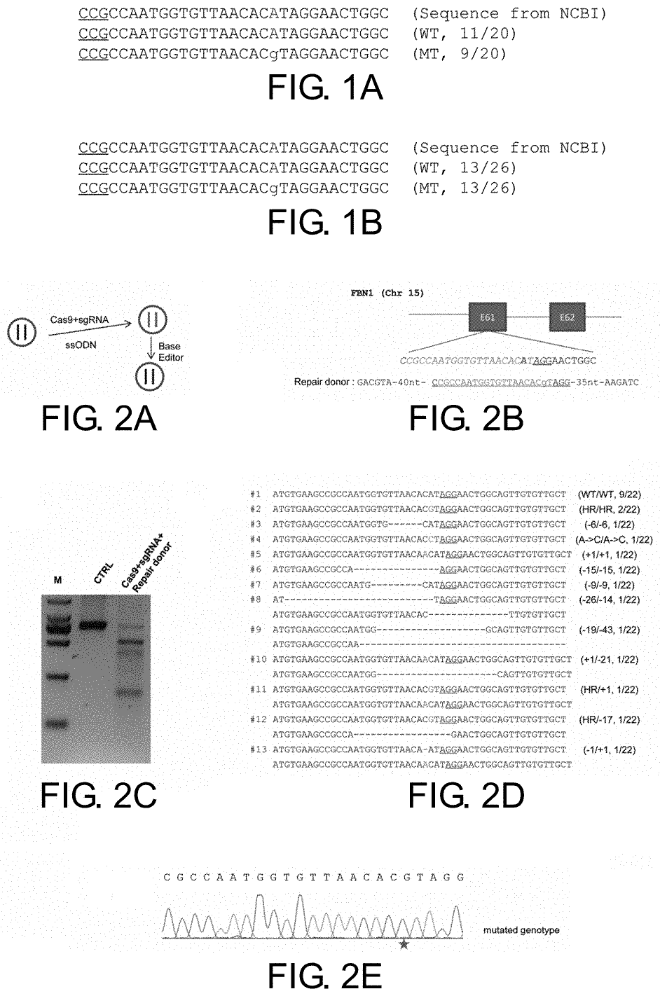

All embryo operations were performed at the Reproductive Medicine Center of the Third Affiliated Hospital of Guangzhou Medical University. The experiment has been approved by the ethics committee of the hospital, and patients who donated eggs and sperms have signed an informed consent. In this experiment, FBN1^{T7498C} heterozygous mutation patients were obtained and their genotypes were identified by blood and semen in the experiment (FIG. 1). The used eggs were all immature eggs of patients with reproductive disorders and matured by being cultured in vitro. The mutated sperms were bound to the eggs by ICSI to obtain mutated zygotes.

2.5 Embryo Repair Operations

When the zygotes were observed to be in the 2PN stage, the base editing mRNA with volume of approximately 0.2 μL, BE3 used in this embodiment, and the sgRNA cytoplasm used for repair were injected into the zygotes by micromanipulation, and the concentrations of BE3 and sgRNA were 100 ng/μL and 50 ng/μL respectively. The treated embryonic cells were further cultured in a tri-gas incubator for three days (FIG. 7).

2.6 Embryo Amplification and Identification

Single-cell amplification of the collected embryos was performed by using a reagent Vazyme, N601-01. The amplified genome was diluted 100 times to amplify target fragments to detect the efficiency of mutation (FIG. 8). Primers for amplifying the target fragments were shown in Table 3.

2.7 Determination of Editing efficiency by Deep Sequencing

To further confirm the editing efficiency, the target fragments were detected by means of PE150 high-throughput sequencing. Three control embryos and all seven repaired embryos were selected. It was found that the three embryos in a control group were of heterozygous genotypes, and the seven repaired embryos were all of normal genotypes, which proves that the base editor can efficiently implement the target site repair.

2.8 Detection of Off-Target Sites of Embryos

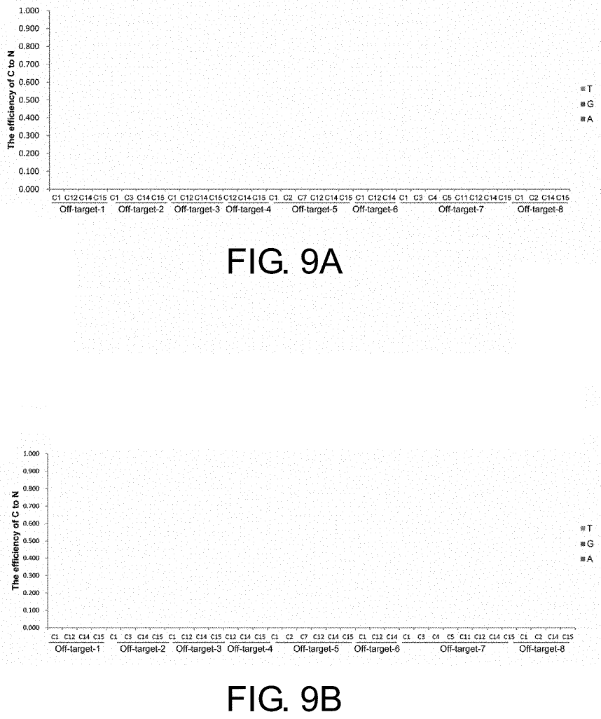

To ensure the safety of the base editor, the off-target detection of three control embryos and all repaired embryos was performed. The results showed (FIG. 9) that no obvious off-target phenomenon was found in the repaired embryos.

1. A kit for efficiently repairing an FBN1^{T7498C} mutation, the kit comprising a base editor and a repair re-sgRNA directed to an FBN1^{T7498C} site.

2. The kit for efficiently repairing an FBN1^{T7498C} mutation according to claim 1, wherein the base editor is one of BE3, YE1-BE3, YE2-BE3 or YEE-BE3.

3. The kit for efficiently repairing an FBN1^{T7498C} mutation according to claim 1, wherein a sequence of the repair re-sgRNA directed to the FBN1^{T7498C} site is SEQ ID NO. 3.

4. A combination for making a mutation and repairing the mutation, the combination comprising:
at least one of a mutated mt-sgRNA designed according to an FBN1^{T7498C} site and a correspondingly mutated ssODN, a repair re-sgRNA directed to the FBN1^{T7498C} site, and a base editor.

5. A method for repairing a mutation by using base editing, the method comprising:
in an FBN1^{T7498C}-containing mutated cell, using a repair re-sgRNA directed to an FBN1^{T7498C} site to guide a base editor to a mutation site to perform base editing repair and collect transfected cells.

6. The method for repairing a mutation by using base editing according to claim 5, wherein the FBN1^{T7498C}-containing mutated cell is an HEK293T cell.

7. The method for repairing a mutation by using base editing according to claim 5, wherein a method for constructing the FBN1^{T7498C}-containing mutated cell comprising:
designing a mutated mt-sgRNA and a correspondingly mutated ssODN according to the FBN1^{T7498C} site;
constructing an expression vector of the mt-sgRNA, allowing a Cas9 protein and the transcribed mt-sgRNA in vitro to form an RNP to be bound to the ssODN and using the expression vector to electrotransfect HEK293T cells, and performing flow sorting on single cells and identifying an FBN1^{T7498C}-containing mutated cell line.

8. The method for repairing a mutation by using base editing according to claim 5, wherein the repair re-sgRNA directed to the FBN1^{T7498C} site is obtained by designing the repair re-sgRNA according to the FBN1^{T7498C} site and constructing a U6 activated and/or T7 activated expression vector.

9. The method for repairing a mutation by using base editing according to claim 7, wherein a sequence of the mt-sgRNA is SEQ ID NO. 1, a sequence of the ssODN is SEQ ID NO. 2, and a sequence of the re-sgRNA is SEQ ID NO. 3.
